(19) United States
(12) Patent Application Publication
(10) Pub. No.: US 2014/0309309 A1
(43) Pub. Date: Oct. 16, 2014

(54) COSMETIC FORMULATION

(71) Applicant: EVONIK INDUSTRIES AG, Essen (DE)

(72) Inventors: Mike Farwick, Essen (DE); Matthias Mentel, Dortmund (DE); Tim Köhler, Dorsten (DE)

(73) Assignee: EVONIK INDUSTRIES AG, Essen (DE)

(21) Appl. No.: 14/355,230

(22) PCT Filed: Oct. 23, 2012
(86) PCT No.: PCT/EP2012/070909

§ 371 Date: Apr. 30, 2014

(30) Foreign Application Priority Data
Oct. 31, 2011 (DE) 102011085497.5

(51) Int. Cl. A61K 8/34; A61Q 19/08; A61K 8/42

(52) U.S. Cl. CPC A61K 8/345; A61K 8/42; A61Q 19/08
USPC 514/629

(57) ABSTRACT

The invention relates to a cosmetic formulation and to its use.

### FIELD OF THE INVENTION

The invention relates to a cosmetic formulation and to its use.

A formulation is to be understood as meaning a preparation comprising an active ingredient and at least one auxiliary. The auxiliary can be for example a solvent.

A cosmetic formulation is accordingly composed such that it can be applied to the human body on account of its low toxicity and its physical properties (for example viscosity) in accordance with its particular purpose.

### PRIOR ART

EP 2 051 691 B1 describes a composition which comprises at least two ceramides. Ceramides consist of a long-chain sphingoid base, the amino group of which is acylated with a fatty acid. The alkyl chain length, the degree of desaturation and the degree of hydroxylation can vary in each case. The composition described in the application is used for treating dry skin.

EP 0 994 888 B1 describes a process for producing phytosphingosine-based ceramides.

### DESCRIPTION OF THE INVENTION

It was an object of the invention to provide a new cosmetic formulation, or to use a cosmetic formulation for a new cosmetic purpose.

The object is achieved by a cosmetic formulation according to claim 1.

None of the compositions described above has the combination claimed in claim 1.

Ageing skin exhibits, inter alia, a loss in elasticity. Gravity brings about a change in shape under the intrinsic weight. Cosmetic problems resulting from this effect manifest themselves for example in pendulous breasts, drooping eyelids and sagging facial skin.

Surprisingly, it has been found that the claimed combination of N-acyl-phytosphingosine and 1,2-pentanediol can be processed significantly better to give cosmetic formulations than combinations of N-acyl-phytosphingosine and other solvents. The claimed combination of N-acyl-phytosphingosine and 1,2-pentanediol results in a lifting of existing formulation restrictions.

Furthermore, the combination of N-acyl-phytosphingosine and 1,2-pentanediol makes it possible to produce cosmetic formulations which are particularly well suited for use on the skin.

In a preferred embodiment, the N-acyl-phytosphingosine is N-hexanoyl-phytosphingosine. A particularly good cosmetic effect as regards the skin was able to be achieved here.

In a further embodiment, the formulation comprises no further ceramides besides the N-acyl-phytosphingosine. Surprisingly, it has been found that very good cosmetic effects could be achieved just with N-acyl-phytosphingosine as the sole ceramide.

In one embodiment, the N-acyl-phytosphingosine is present in the formulation in a concentration of 0.02% by mass to 2% by mass, based on the total formulation. Preferably, the N-acyl-phytosphingosine is present in the formulation in a concentration of 0.1% by mass to 1% by mass, based on the total formulation. Particularly good cosmetic effects were able to be achieved for these mass concentrations.

In one embodiment, the 1,2-pentanediol is present in the formulation in a concentration of 0.2% by mass to 15% by mass, based on the total formulation. Preferably, the 1,2-pentanediol is present in the formulation in a concentration of 0.5% by mass to 10% by mass, based on the total formulation. Particularly preferably, the 1,2-pentanediol is present in the formulation in a concentration of 1% by mass to 5% by mass, based on the total formulation. Particularly good cosmetic effects were able to be achieved for these mass concentrations.

In a further embodiment, the ratio of N-acyl-phytosphingosine to 1,2-pentanediol is in the range from 1:100 to 1:1. Preferably, the ratio of N-acyl-phytosphingosine to 1,2-pentanediol is in the range from 1:25 to 1:1. Particularly preferably, the ratio of N-acyl-phytosphingosine to 1,2-pentanediol is in the range from 1:15 to 1:1. The ratio of N-acyl-phytosphingosine to 1,2-pentanediol is important for the formulation since it influences inter alia the application properties, such as, for example, the viscosity of the formulation or the ability to be absorbed by the skin.

Suitable auxiliaries present in the cosmetic formulation are, for example, moreover, emollients, emulsifiers and surfactants, thickeners/viscosity regulators/stabilizers, UV light protection filters, antioxidants, hydrotropes (or polyols), solids and fillers, film formers, pearlescent additives, deodorant and antiperspirant active ingredients, insect repellents, self-tanning agents, preservatives, conditioners, perfumes, dyes, biogenic active ingredients, care additives, superfatting agents and further solvents. In particular, the cosmetic formulation comprises at least one further biogenic active ingredient; biogenic active ingredients are to be understood as meaning, for example, tocopherol, tocopherol acetate, tocopherol palmitate, ascorbic acid, polyphenols, deoxyribonucleic acid, coenzyme Q10, retinol, AHA acids, amino acids, hyaluronic acid, alpha-hydroxy acids, isoflavones, polyglutamic acid, creatine (and creatine derivates), guanidine (and guanidine derivates), pseudoceramides, essential oils, peptides, protein hydrolysates, plant extracts, bisabolol, allantoin, panthenol, phytantriol, idebenone, liquorice extract, glycyrrhizidine and idebenone, scleroglucan, β-glucan, santalbic acid and vitamin complexes. Examples of plant extracts are horse chestnut extract, camomile extract, rosemary extract, black and red currant extract, birch extract, rosehip extract, algae extracts, green tea extract, aloe extract, ginseng extract, gingko extract, grapefruit extract, calendula extract, cucumber extract, camphor, thyme extract, mangosteen extract, cystus extract, terminalia arjuna extract, oat extract, oregano extract, raspberry extract, strawberry extract, etc. The biogenic active ingredients can also include the so-called barrier lipids, examples of which are ceramides, phytosphingosine and derivatives, sphingosine and derivatives, sphinganin and derivatives, pseudoceramides, phospholipids, lysophospholipids, cholesterol and derivatives, cholesteryl esters, free fatty acids, lanolin and derivatives, squalane, squalene and related substances. Within the context of the invention, the biogenic active ingredients also include anti-acne ones, such as, e.g. benzoyl peroxide, phytosphingosine and derivatives, niacinamide hydroxybenzoate, nicotinaldehyde, retinoic acid and derivatives, salicylic acid and derivatives, citronellic acid etc. and anti-cellulite ones such as e.g. xanthine compounds such as caffeine, theophylline, theobromine and aminophylline, carnitine, carnosine, salicyloyl phytosphingosine, phytosphingosines, santalbic acid etc., as well as antidandruff agents such as, for example, salicylic acid and derivatives, zinc pyrithione, selenium sulphide, sulphur, cyclopiroxolamine, bifonazole, climbazole, octopirox and actirox etc. as well as astringents such as e.g. alcohol, aluminium derivatives, gallic acid, pyroxidine salicylate, zinc salt such as e.g. zinc sulphate, acetate, chloride, lactate, zirconium chlorohydrates etc. The biogenic active ingredients can likewise include bleaches such as kojic acid, arbutin, vitamin C and derivatives, hydroquinone, turmeric oil, creatinine, sphingolipids, niacinamide, etc.

In a further embodiment, the cosmetic formulation comprises partition modifiers such as, for example benzyl alcohol, alpha-bisabolol, cetyl alcohol, chitosan, decanol, decyl methyl sulphoxide, diethylene glycol monoethyl ether, dimethylformamide, dimethylacetamide, dimethyl sulphoxide, EDTA, ethylene glycol, 2-pyrrolidone, N-methyl-2-pyrrolidone, 1-dodecylazacycloheptan-2-one, 4-decyloxazolidin-2-one, ethanol, glycerol, lauric acid, lauryl alcohol, lecithin, urea, oleic acid, linoleic acid, linolenic acid, dimethyl isosorbide, isopropyl myristate, propylene glycol, sodium lauryl sulphate, cetyltrimethylammonium bromide, dodecyl betaine, terpenes such as d-limonene, N-methylformamide, stearic acid, polyethoxyethylenes, limonene oxides, hydrophobins, 3-methoxy-3-methyl-1-butanol, 3-methyl-1,3-butanediol, 1,3-propanediol, glyceryl monopalmitate, dodecanoyl acetate, cetyl ethylhexanoate, stearyl ethylhexanoate, ethyl lactate, isobutyl lactate, triethyl acetyl citrate, phytantriol, salicylic acid and derivatives thereof, ascorbic acid and derivatives thereof, butylene glycol, thiazone, sodium dodecyl sulphate, nerolidol and 1,8-cineol, glycolipids, medium-chain triglycerides, branched-chain fatty alcohols such as octyldodecanol, taurine, phospholipids, 1,2-pentylene glycol and C8/C10-glycerol partial esters (esterification product of glycerol with a substoichiometric mixture comprising n-octanoic acid and n-decanoic acid), in particular C8/C10-glycerol partial esters, as described for example in PCT/EP2011/056616. Particularly good values can be achieved using this additional auxiliary.

In a further embodiment, the cosmetic formulation comprises C12-15 alkyl benzoate, in particular TEGOSOFT® TN. Particularly good values were able to be achieved using this additional auxiliary.

In a further embodiment, the cosmetic formulation comprises PPG-13 myristyl ether, in particular TEGOSOFT® APM. Particularly good values were able to be achieved using this additional auxiliary.

In a further embodiment, the cosmetic formulation comprises octyl dodecanol, in particular TEGOSOFT® G20. Particularly good values were able to be achieved using this additional auxiliary.

Besides the cosmetic formulation itself, various uses of the same are also claimed.

The use of an above-described formulation for application to the skin is claimed.

Further claimed is the use of an above-described formulation for reducing skin slackening. It was able to be shown that skin slackening could be noticeably reduced by using a formulation described above.

Further claimed is the use of an above-described formulation for plumping up the skin. Noticeable improvements could also be achieved as regards plumping up.

A lifting effect can be achieved as a result of a combination of plumping up and reduction in slackening.

In a preferred variant of the use, the cosmetic formulation is applied to the face. Here, very good results were able to be achieved particularly in the region of the eyelids and the cheeks.

In a further preferred variant of the use, the cosmetic formulation is applied to the breast. Here too very good results were able to be achieved.

Besides the lifting effect, a more targeted shaping can also take place.

The use of a cosmetic formulation comprising an N-acyl-phytosphingosine for reducing skin slackening is also claimed. The auxiliary present in the cosmetic formulation can be for example a solvent and thus for example 1,2-pentanediol.

In a preferred variant of the use, the auxiliary is 1,2-pentanediol.

Furthermore, the use of a cosmetic formulation comprising N-hexanoyl-phytosphingosine for reducing skin slackening is claimed. Here too, the auxiliary present in the cosmetic formulation can be for example a solvent and thus for example 1,2-pentanediol. In a preferred variant of the use, the auxiliary is 1,2-pentanediol.

Further claimed is the use of a cosmetic formulation comprising N-hexanoyl-phytosphingosine as the sole ceramide for reducing skin slackening. Here too, the auxiliary present in the cosmetic formulation can be for example a solvent and thus for example 1,2-pentanediol.

In a preferred variant of the use, the auxiliary is 1,2-pentanediol.

The use described in the last three paragraphs takes place preferably in the face and breast area.

The use of a cosmetic formulation comprising N-hexanoyl-phytosphingosine for cosmetic breast enlargement is also claimed. Here too, the auxiliary present in the cosmetic formulation can for example be a solvent and thus for example 1,2-pentanediol. In a preferred variant of the use, the auxiliary is 1,2-pentanediol.

The examples below describe the present invention by way of example without any intention of limiting the invention, the scope of application of which arises from the entire description and the claims, to the embodiments specified in the examples.

### EXAMPLES

### Example 1

### Investigation of the Influence of N-hexanoyl-phytosphingosine on the Gene Expression in Human Dermal Fibroblasts

The present example investigates the effect of N-hexanoyl-phytosphingosine on the gene expression in fibroblasts (normal human dermal fibroblasts, NHDFs).

For this, firstly primary human dermal fibroblasts (human dermal fibroblasts derived from neonatal skin, HDF), cryopreserved, Lifeline Cell Technology, acquired via CellSystems® Biotechnologie Vertrieb GmbH, in Minimum Essential Medium (MEM) with Earle's salts (EMEM) (PAA Laboratories GmbH) were cultivated with the addition of 10% foetal bovine serum (FBS, Invitrogen Ltd, UK), 1% nonessential amino acids (NEAA) (100×) (PAA Laboratories GmbH), 1% L-glutamine (100×) (Invitrogen Ltd) and 1% penicillin/streptomycin (5000 U/mL penicillin and 5000 μg/mL streptomycin/mL, (Invitrogen Ltd) at 37° C. and 5% CO₂. For gene expression studies, the cells were sown out in 6-well plates and cultivated to the point of subconfluence (maximum 60%).

Then, the medium was removed from the cells and replaced by fresh medium containing N-hexanoyl-phytosphingosine. The vehicle used was methanol, the end concentration of the vehicle in the medium being 0.05% (v/v). The end concentration of N-hexanoyl-phytosphingosine in the medium was 5 μM. As the control, the cultivation was carried out without active ingredient, only with medium (vehicle). All of the cultivations were performed twice (with cells from two different donors).

After cultivation for 24 hours, the medium was removed and the cells were lysed by adding RNeasy Lysis Buffer (Qiagen). Isolation of whole-RNA was carried out in accordance with manufacturer's instructions. Combined, the whole-RNA was isolated by means of RNeasy Mini Kit (Qiagen). The RNA concentration was determined by spectrophotometry using a SmartSpec Plus (Biorad). Purity and integrity of the RNA were determined using Agilent 2100 Bioanalyzer with a 6000 Nano LabChip reagent set (Agilent Technologies, Inc.). RNA samples were stored at −80° C. until further processing.

Analysis of the gene expression was carried out using Affymetrix HGU133 plus 2.0 GeneChips (Affymetrix), with 2 μg of whole-RNA being used. For this, the RNA of the two different donor cultures was pooled. Expression analyses including standard data evaluation were carried out in accordance with manufacturer's instructions (AFFYMETRIX, INC.). Surprisingly, it has been found that the treatment of the fibroblasts with N-hexanoyl-phytosphingosine led to the modified expression of a series of genes to which is attributed the central importance for strengthening the extracellular matrix of the dermis and consequently for the tightening, the elasticity and the firming of the skin, as a result of which skin slackening can be significantly reduced, and the skin can be plumped up. The genes and the extent of their regulation by N-hexanoyl-phytosphingosine are shown in Table 1.

TABLE 1
Selection of at least 1.4-fold up- or down-regulated genes in
human dermal fibroblasts 24 h after application of 5 μM of
N-hexanoyl-phytosphingosine. All of the values refer to the
comparison with vehicle-treated fibroblasts.

| | | Regulation of the |
|---|---|---|
| | | gene expression |
| | | by N-hexanoyl- |
| Gene | Protein | phytosphingosine |
| IGF1 | insulin-like growth factor 1 | +2.0 |
| ACAN | aggrecan | +1.7 |
| LEP | leptin | +2.2 |
| COL11A1 | collagen, type XI, alpha 1 | +2.2 |
| HAPLN1 | hyaluronan and proteoglycan | +4.1 |
| | link protein | |
| ITGA2 | integrin alpha-2 | +1.6 |
| FGF18 | fibroblast growth factor 18 | +2.2 |
| COL10A1 | collagen, type X, alpha 1 | +3.2 |
| MMP3 | matrix metallopeptidase 3 | −1.4 |

On the one hand, a series of genes was regulated which have a central importance for the formation and strengthening of the dermal extracellular matrix. Including ACAN, which codes for a proteoglycan and therefore a component of the extracellular matrix. COL10A1 and COL11A1 code for different collagen subunits. Consequently, the formation of collagen fibrils is positively influenced. The ITGA2 gene product is a constituent of the receptor for laminin, collagen, fibronectin and other important constituents of the extracellular matrix. It is attributed particular importance for the organization of the newly formed extracellular matrix. HAPLN1 codes for a bridging protein which stabilizes aggregates of proteoglycan monomers and hyaluronic acid in the extracellular matrix. MMP3 codes for an enzyme which degrades various molecules of the extracellular matrix. Since the expression of MMP3 is reduced by N-hexanoyl-phytosphingosine, the extracellular matrix is consequently protected against degradation. Finally, IGF1, LEP and FGF18 code for growth factors or hormone-like molecules which are attributed important signal transduction functions which for their part have a positive influence on the structure of the dermal matrix, and therefore the firmness, compactness and elasticity of the skin.

Example 1 therefore clearly shows that N-hexanoyl-phytosphingosine is exceptionally well suited for reducing skin slackening and for plumping up the skin.

### Example 2

### Investigation of the Influence of N-hexanoyl-phytosphingosine on the Gene Expression in Adipocytes

This example investigated the effect of N-hexanoyl-phytosphingosine on the gene expression in differentiated human adipocytes.

Cells, media and reagents were acquired from PromoCell. The cultivation was carried out in accordance with the protocol from PromoCell. Unless stated otherwise, the cells were cultivated at 37° C. and 5% CO₂. Firstly, proliferating subcutaneous primary human white preadipocytes (HWP) from two different donors were cultivated in a special preadipocyte growth medium until complete confluence was reached. The medium was then exchanged for a special differential medium, and the cells were incubated with this medium for 72 hours. The medium was then exchanged for a further medium (Adipocyte Nutrition Medium) in order to conclude the differentiation process. The cells were further incubated with this medium for twelve days, with media exchange being carried out every three days. Ultimately, the medium was removed from the cells and replaced by fresh medium containing N-hexanoyl-phytosphingosine. The vehicle used was methanol, the end concentration of the vehicle in the medium being 0.05% (v/v). End concentration of N-hexanoyl-phytosphingosine in the medium was 2.5 μM. As the control, the cultivation was carried out without active ingredient, only with medium (vehicle). All of the cultivations were performed twice (with cells from two different donors).

After cultivation for six hours, the medium was removed and the cells were lysed by adding RNeasy Lysis Buffer (Qiagen). Isolation of whole-RNA was carried out in accordance with manufacturer's instructions. Combined, the whole-RNA was isolated by means of RNeasy Mini Kit (Qiagen). The RNA concentration was determined by spectrophotometry using a SmartSpec Plus (Biorad). Purity and integrity of the RNA were determined using Agilent 2100 Bioanalyzer with a 6000 Nano LabChip reagent set (Agilent Technologies, Inc.). RNA samples were stored at −80° C. until further processing.

Analysis of the gene expression was carried out using Affymetrix HGU133 plus 2.0 GeneChips (Affymetrix), with 2 μg of whole-RNA being used. For this, the RNA of the two different donor cultures was pooled. Expression analyses including standard data evaluation were carried out in accordance with manufacturer's instructions (Affymetrix.).

Surprisingly, it has been found that the treatment of the differentiated adipocytes with N-hexanoyl-phytosphingosine led to the modified expression of a series of genes to which a central importance is attributed for the biosynthesis of storage fats in the subcutaneous fatty tissue of the skin. The observed regulation pattern points to an increased biosynthesis of storage fat (lipogenesis). Such a mechanism can explain the effects observed in the in vivo study (see below), i.e. the smoothing of the skin and the reduction in the sagging of the skin. The skin is plumped up to a certain extent by newly formed fat.

The genes and the extent of their regulation by N-hexanoyl-phytosphingosine are shown in Table 2.

TABLE 2
Selection of at least 1.4-fold up- or down-regulated genes
in differentiatedhuman adipocytes 6 h after application
of 2.5 μM N-hexanoyl-phytosphingosine. All values
refer to the comparison with vehicle-treated adipocytes.

| | | Regulation of the |
|---|---|---|
| | | gene expression |
| | | by N-hexanoyl- |
| Gene | Protein | phytosphingosine |
| ADIPOQ | adiponectin | +2.2 |
| LEP | leptin | +2.7 |
| LEPR | leptin receptor | +4.1 |
| PPARG | peroxisome proliferator | +2.5 |
| | activated receptor, gamma | |
| PPARD | peroxisome proliferator | +1.7 |
| | activated receptor, delta | |
| CEBPB | CAAT/enhancer binding | +2.1 |
| | protein (C/EBP), beta | |
| LIPC | lipase | −2.5 |
| FAS | fatty acid synthase | +1.4 |
| ACACA | acetyl-coenzyme | +1.5 |
| | Acarboxylase alpha | |
| THBS1 | thrombospondin 1 | +5.9 |

As regards the genes of fat metabolism, LIPC, FAS and ACACA are noticeable in particular. The first codes for a lipase. These enzymes generally catalyse the degradation of triglycerides. Since the gene LIPC in the present example was down-regulated by the test substance, a degradation of subcutaneous fatty tissue can thus be postulated. FAS codes for the enzyme fatty acid synthase, ACACA for the acetyl-coenzyme A carboxylase. Both are key enzymes for the triglyceride biosynthesis. Since both corresponding genes were up-regulated by the test substance, a positive effect on the degradation of fatty tissue can also be postulated here. CEBPB, PPARG and PPARG code for transcription factors which are known inter alia for regulating the lipid biosynthesis. Here too, the regulation pattern again points to a stimulation of fat formation (lipogenesis).

Besides these genes of fat metabolism, the expression of a series of genes coding for growth factors or hormone-like substances in the adipocytes was also regulated. Examples here are LEP, LEPR and ADIPOQ. These all have importance for regulating the new formation and strengthening of the dermal extracellular matrix. In this regard, these factors formed by the adipocytes can presumably contribute in vivo via cell communication mechanism to the strengthening of the skin structure.

Consequently, it can also be clearly shown in example 2 that N-hexanoyl-phytosphingosine is exceptionally well suited to reducing skin slackening and for plumping up the skin.

### Example 3

### Reduction in the Sagging and the Roughness and Increasing the Echogenicity of the Skin by N-hexanoyl-phytosphingosine

The improvement in the parameters sagging of the skin, roughness of the skin and echogenicity of the skin by topical application of N-hexanoyl-phytosphingosine in formulation was demonstrated in a human study.

The panel comprises 60 healthy female subjects of Caucasian origin up to 70 years of age who were in their postmenopausal phase (average age 59.4 years). The formulations were applied on the face. Vehicle formulation (vehicle) and N-hexanoyl-phytosphingosine formulation (N-hexanoyl-phytosphingosine) was each applied by 30 subjects. In the case of the non-inventive vehicle formulation, which serves as comparison formulation, the 0.2% by mass of N-hexanoyl-phytosphingosine were replaced by an additional 0.2% by mass of water.

Application was carried out twice daily, morning and evening, over a period of twelve weeks. Measurement points were before the start of application (T0), after four weeks (T4), after eight weeks (T8) and after twelve weeks (T12). A period over four weeks is accordingly referred to as T4-T0.

The composition of the formulations is shown in Table 3 below:

TABLE 3
Test formulation. Data in percent by mass.

| | | | N-hexanoyl- |
|---|---|---|---|
| | | | phytosphin- |
| Phase | Raw material | Vehicle | gosine |
| A | TEGINACID ® C (Ceteareth-25) | 2.00 | 2.00 |
| | ABIL ® Care 85 (Bis-PEG/PPG- | 1.00 | 1.00 |
| | 16/16 PEG/PPG16/16 Dimethicone; | | |
| | Caprylic/Capric Triglyceride) | | |
| | TEGO ® Alkanol 1618 (Cetearyl | 5.00 | 5.00 |
| | Alcohol) | | |
| | Stearin (Stearic acid (and) palmitic | 0.50 | 0.50 |
| | acid) | | |
| | TEGOSOFT ® APM (PPG-13 | 4.83 | 4.83 |
| | Myristyl Ether) | | |
| | TEGOSOFT ® G20 (Octyl | 4.83 | 4.83 |
| | Dodecanol) | | |
| | TEGOSOFT ® TN (C12-15 Alkyl | 4.83 | 4.83 |
| | Benzoate) | | |
| | N-hexanoyl-phytosphingosine | | 0.2 |
| B | Water | 71.80 | 71.60 |
| | Glycerol (Glycerin) | 2.54 | 2.54 |
| C | TEGO ® Carbomer 134 (Carbomer) | 0.15 | 0.15 |
| | TEGOSOFT ® APM/G20/TN 1:1:1 | 0.60 | 0.60 |
| D | 1,2-pentanediol (Pentylene Glycol) | 1.80 | 1.80 |
| Z | Euxyl K220 (Methylisothiazolinone, | 0.12 | 0.12 |
| | Ethylhexylglycerin, Water) | | |
| | NaOH 10% (Sodium Hydroxide) | pH 5.5 | pH 5.5 |

To prepare the formulations, customary formulation processes known to the person skilled in the art were used.

The measurements were performed in a temperature- and humidity-controlled room (24±2° C., 50±10 relative humidity). The subjects were requested to apply no product up to twelve hours prior to measurement and to wash their face three hours before measurement.

The sagging of the skin was assessed by a dermatologist by means of visual and tactile evaluation by reference to a 4-point scale (0=absent, 1=minimum, 2=moderate, 3=maximum).

The roughness of the skin was determined by means of Primos Pico optical 3D-Sensor (GF Messtechnik GmbH). Primos Pico utilizes a digital band projection based on micromirrors which permit rapid and highly precise data capture. Bands are projected onto the surface of the skin and the projection is recorded under a defined triangulation angle by means of a CCD camera. The topography of the measured object is calculated by the position of the bands and the grey scales of all registered image points. The measurement field is 40×30 mm² with a lateral resolution of 63 μm, a vertical resolution of ≧4 μm and an accuracy of ≦6 μm. The data is captured within <70 ms. The average roughness Sa was determined; this reflects the arithmetic mean of the skin roughness in the recorded image.

The echogenicity of the skin was measured using a DermaScan® C ultrasound scanner. The instrument is based on the physical principle of the ultrasound emission of a transducer. If an ultrasound beam hits structurally different layers of the skin, it is partly transmitted and partly reflected. In this way, echo soundwaves with different amplitudes are generated. Their intensity is detected by a microprocessor. DermaScan® C Version 3 (Cortex Technology, Denmark) is a high resolution scanner which emits high frequency ultrasound (20 MHz). This frequency permits the observation of tissue structures down to a depth of 15 mm with 60 μm axial and 200 μm lateral resolution. The echogenicity was recorded in the B-scan mode and given in %. The instrument measures the thickness of the skin layers. The intensity of the soundwaves is dependent on the tissue thickness. Younger skin is essentially thicker than aged skin. The effectiveness of a tissue-thickened treatment is indicated by an increase in skin thickness.

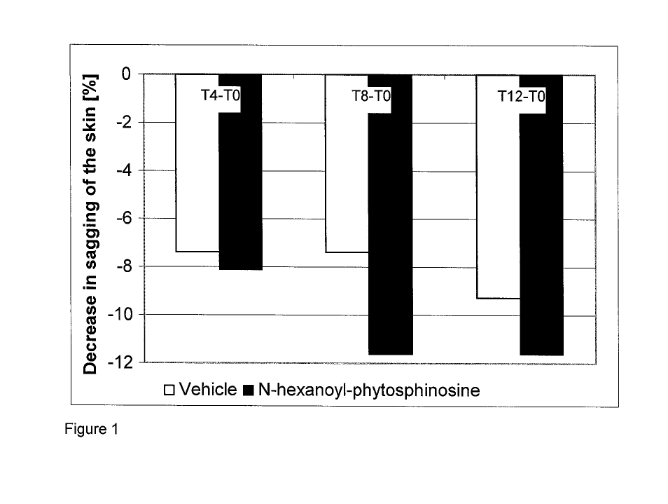

FIG. 1 shows the decrease in sagging of the skin.

Over a period of twelve weeks (T12-T0) a steady reduction in the sagging of the skin was observed both in the vehicle group and also in the N-hexanoyl-phytosphingosine group, with the decrease in sagging of the skin being in each case more marked in the N-hexanoyl-phytosphingosine group at all time points.

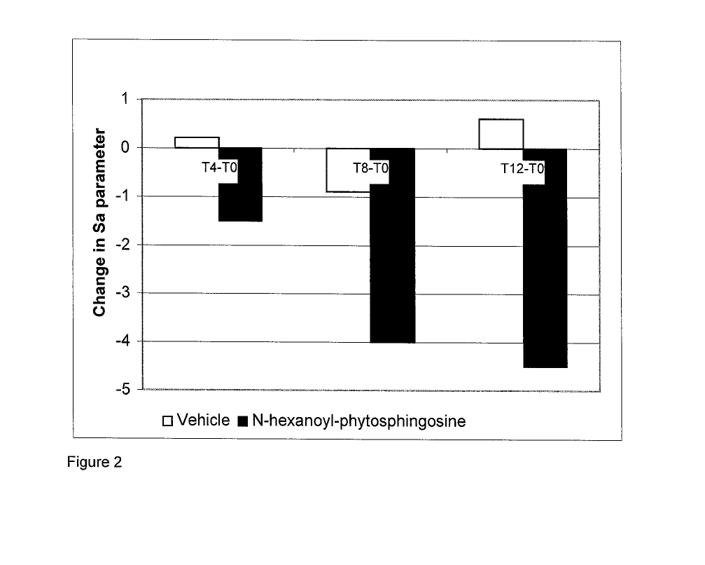

FIG. 2 shows the change in skin roughness (Sa parameter).

The skin roughness in the N-hexanoyl-phytosphingosine group was steadily further reduced over the entire study period whereas no improvement in skin roughness was attained in the vehicle group.

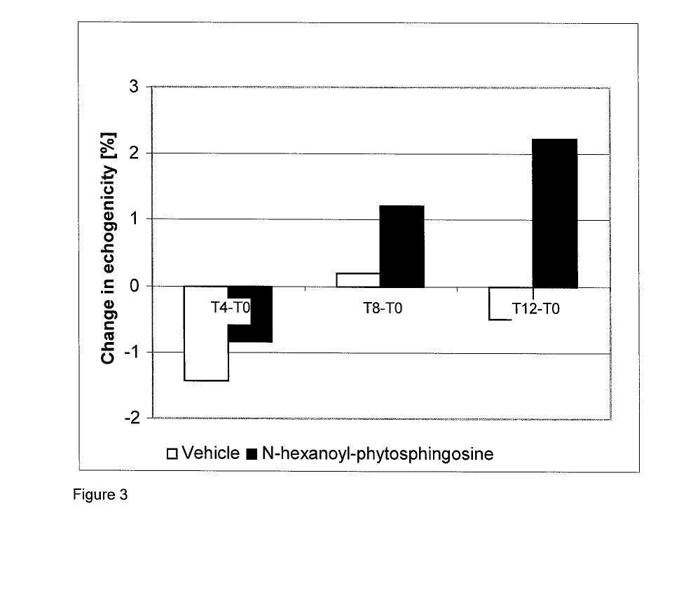

FIG. 3 shows the change in the echogenicity of the skin.

A slight decrease in echogenicity was detected after four weeks (T4-T0) both in the vehicle group and in the N-hexanoyl-phytosphingosine group. Whereas treatment with vehicle formulation exhibited no improvement compared with the starting value as the study progressed, the echogenicity in the N-hexanoyl-phytosphingosine group was steadily improved.

These results, shown in FIGS. 1 to 3, show that by using N-hexanoyl-phytosphingosine in the formulation, it is possible to achieve a decrease in the sagging of the skin, a decrease in skin roughness and an increase in echogenicity of the skin.

### Example 4

### Increasing the Bioavailability of N-hexanoyl-phytosphingosine by Formulation with 1,2-pentanediol

The increase in the skin penetration of N-hexanoyl-phytosphingosine from a cosmetic formulation as a result of adding 1,2-pentanediol was shown in the course of a percutaneous absorption study on a pig skin model. In practice, the study was performed in accordance with a current protocol, as described for example in S. Richert, A. Schrader, K. Schrader, Int. J. Cosmet. Sci. 2003, 25, 5-13.

The investigated formulations are summarized in Table 4 below.

TABLE 4
Test formulations.

| | | | | | TN/G20/ | | |
|---|---|---|---|---|---|---|---|
| | | TN/ | | | APM | | |
| | | G20/ | | | 1:1:1 + | | Paraffin |
| | | APM | | Paraffin | 1,2- | CT + 1,2- | Oil + 1,2- |
| | Raw material | 1:1:1 | CT | Oil | Pentanediol | Pentanediol | Pentanediol |
| A | TEGINACID ® C | 2.00 | 2.00 | 2.00 | 2.00 | 2.00 | 2.00 |
| | (Ceteareth-25) | | | | | | |
| | ABIL ® Care 85 (Bis-PEG/ | 1.00 | 1.00 | 1.00 | 1.00 | 1.00 | 1.00 |
| | PPG-16/16 PEG/PPG16/ | | | | | | |
| | 16 Dimethicone; Caprylic/ | | | | | | |
| | Capric Triglyceride) | | | | | | |
| | TEGO ® Alkanol 1618 | 5.00 | 5.00 | 5.00 | 5.00 | 5.00 | 5.00 |
| | (Cetearyl Alcohol) | | | | | | |
| | Stearin (Stearic acid (and) | 0.50 | 0.50 | 0.50 | 0.50 | 0.50 | 0.50 |
| | palmitic acid) | | | | | | |
| | TEGOSOFT ® TN (C12-15 | 4.83 | | | 4.83 | | |
| | Alkyl Benzoate) | | | | | | |
| | TEGOSOFT ® APM (PPG- | 4.83 | | | 4.83 | | |
| | 13 Myristyl Ether) | | | | | | |
| | TEGOSOFT ® G20 (Octyl | 4.83 | | | 4.83 | | |
| | Dodecanol) | | | | | | |
| | TEGOSOFT ® CT | | 14.5 | | | 14.5 | |
| | (Caprylic/Capric | | | | | | |
| | Triglycerides) | | | | | | |
| | Paraffinum Liquidum (30 | | | 14.5 | | | 14.5 |
| | mPa*s) | | | | | | |
| | N-hexanoyl- | 0.2 | 0.2 | 0.2 | 0.2 | 0.2 | 0.2 |
| | phytosphingosine | | | | | | |
| B | Water | 72.26 | 72.25 | 72.25 | 70.46 | 70.45 | 70.45 |
| | Glycerol (Glycerin) | 3 | 3 | 3 | 3 | 3 | 3 |
| C | TEGO ® Carbomer 134 | 0.20 | 0.20 | 0.20 | 0.20 | 0.20 | 0.20 |
| | (Carbomer) | | | | | | |
| | TEGOSOFT ® Emollient | 0.80 | 0.80 | 0.80 | 0.80 | 0.80 | 0.80 |
| | (vide supra) | | | | | | |
| D | 1,2-pentanediol (Pentylene | — | — | — | 1.80 | 1.80 | 1.80 |
| | Glycol) | | | | | | |
| Z | Euxyl K220 | 0.12 | 0.12 | 0.12 | 0.12 | 0.12 | 0.12 |
| | (Methylisothiazolinone, | | | | | | |
| | Ethylhexylglycerin, Water) | | | | | | |
| | NaOH 10% (Sodium | 0.43 | 0.43 | 0.43 | 0.43 | 0.43 | 0.43 |
| | Hydroxide) | | | | | | |

Data in per cent by mass.

To prepare the formulations, customary formulation processes known to the person skilled in the art were used.

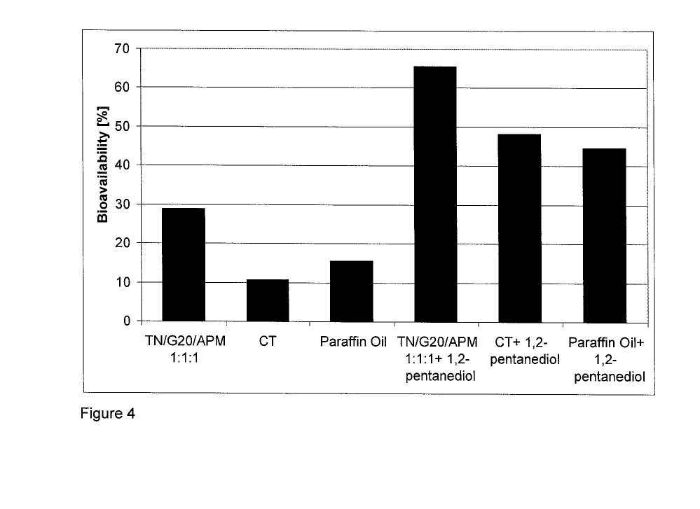

FIG. 4 shows the bioavailability of N-hexanoyl-phytosphingosine from various formulations.

These results show that by using N-hexanoyl-phytosphingosine in combination with 1,2-pentanediol in formulation it is possible to achieve an increase in bioavailability. The bioavailability is an important factor for cosmetic formulations since it gives information about what fraction of the active ingredient present in the formulation is also then in fact taken up by the organism. A high bioavailability thus constitutes a clear advantage. For example, the amount of active ingredient which has to be incorporated into the formulation can be reduced as a result in the case of a high bioavailability.

The results from Example 4 show that the combination of N-hexanoyl-phytosphingosine and 1,2-pentanediol has a clear advantage compared with the corresponding formulations without 1,2-pentanediol.

### Example 5

### Reduction in Skin Slackening on the Upper Arm by Means of N-hexanoyl-phytosphingosine

The reduction in skin slackening on the upper arm, quantifiable by the parameter of echogenicity of the skin, by topical application of N-hexanoyl-phytosphingosine in formulation was demonstrated in a human study.

The panel consisted of healthy female subjects of Caucasian origin up to 70 years old who were in their postmenopausal phase. The echogenicity of the skin on the upper arms of the subjects was at most 60% (DermaScan® C, B-scan mode). The formulations were applied to the upper arms. Vehicle formulation (vehicle) and formulation with different concentrations of N-hexanoyl-phytosphingosine (N-hexanoyl-phytosphingosine) was applied by in each case 16 subjects. In the non-inventive vehicle formulation, which serves as comparison formulation, the corresponding % by mass of N-hexanoyl-phytosphingosine were replaced by additional % by mass of water.

Application was carried out twice daily, morning and evening, over a period of twelve weeks. Measurement points were before the start of application (T0), after four weeks (T4), after eight weeks (T8) and after twelve weeks (T12). A period over four weeks is accordingly referred to as T4-T0.

The composition of the formulations is shown in Table 3 below:

TABLE 5
Test formulations. Data in percent by mass.

| | | | 0.02% | 0.05% |
|---|---|---|---|---|
| | | | N-hexanoyl- | N-hexanoyl- |
| Phase | Raw material | Vehicle | phytosphingosine | phytosphingosine |
| A | TEGINACID ® C (Ceteareth-25) | 2.00 | 2.00 | 2.00 |
| | ABIL ® Care 85 (Bis-PEG/PPG- | 1.00 | 1.00 | 1.00 |
| | 16/16 PEG/PPG16/16 | | | |
| | Dimethicone; Caprylic/Capric | | | |
| | Triglyceride) | | | |
| | TEGO ® Alkanol 1618 (Cetearyl | 5.00 | 5.00 | 5.00 |
| | Alcohol) | | | |
| | Stearin (Stearic acid (and) | 0.50 | 0.50 | 0.50 |
| | palmitic acid) | | | |
| | TEGOSOFT ® APM (PPG-13 | 4.83 | 4.83 | 4.83 |
| | Myristyl Ether) | | | |
| | TEGOSOFT ® G20 (Octyl | 4.83 | 4.83 | 4.83 |
| | Dodecanol) | | | |
| | TEGOSOFT ® TN (C12-15 Alkyl | 4.83 | 4.83 | 4.83 |
| | Benzoate) | | | |
| B | Water | 69.84 | 69.82 | 69.79 |
| | Glycerol (Glycerin) | 2.50 | 2.50 | 2.50 |
| C | TEGO ® Carbomer 134 | 0.15 | 0.15 | 0.15 |
| | (Carbomer) | | | |
| | TEGOSOFT ® APM/G20/TN 1:1:1 | 0.60 | 0.60 | 0.60 |
| D | N-hexanoyl-phytosphingosine | | 0.02 | 0.05 |
| | 1,2-pentanediol (Pentylene | 3.80 | 3.80 | 3.80 |
| | Glycol) | | | |
| Z | Euxyl K220 | 0.12 | 0.12 | 0.12 |
| | (Methylisothiazolinone, | | | |
| | Ethylhexylglycerin, Water) | | | |
| | Sodium Hydroxide (10% in water) | pH 5.5 | pH 5.5 | pH 5.5 |

To prepare the formulations, customary formulation processes known to the person skilled in the art were used.

The measurements were performed in a temperature- and humidity-controlled room (24±2° C., 50±10 relative humidity). The subjects were asked to apply no product up to twelve hours before measurement and to no longer wash their arms from three hours before measurement.

The skin slackening was measured by measuring the echogenicity of the skin by means of a DermaScan® C ultrasound scanner. The echogenicity was recorded in the B-scan mode and given in %. The effectiveness of a treatment to reduce skin slackening is indicated by an increase in echogenicity in [%], and thus associated with an increase in skin thickness.

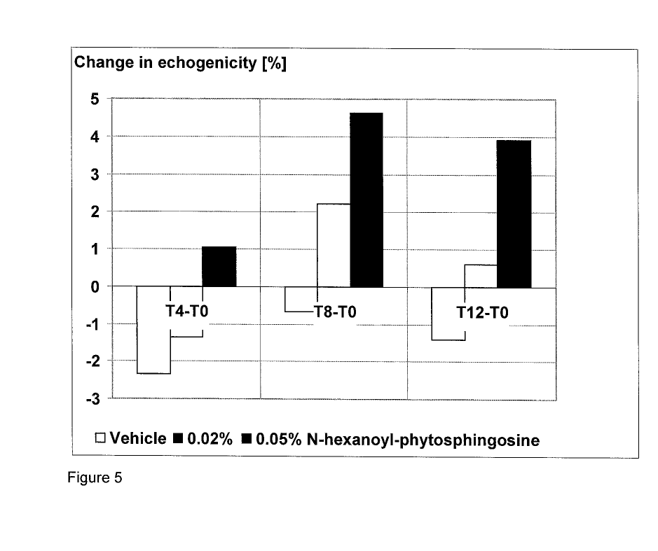

FIG. 5 shows the increase in skin thickness on the upper arm.

Over a period of four, eight and twelve weeks, in each case an increase in the echogenicity of the skin as a result of treatment with 0.05% N-hexanoyl-phytosphingosine, compared with the starting value, was observed. This increase was less marked in the case of a treatment with 0.02% N-hexanoyl-phytosphingosine and could only be measured after a treatment time of eight weeks. As a result of the treatment with vehicle formulation, no increase in the echogenicity of the skin was achieved, but instead, at all time points, in each case a decrease in echogenicity of the skin.

This result shows that by using 0.02% or 0.05% of N-hexanoyl-phytosphingosine in formulation it is possible to achieve an increase in the echogenicity on the upper arm, compared to the vehicle formulation used. The increase in the echogenicity on the upper arm is synonymous with a decrease in skin slackening on the upper arm.

1. A cosmetic formulation comprising:
an N-acyl-phytosphingosine; and
1,2-pentanediol.

2. The cosmetic formulation according to claim 1, wherein said N-acyl-phytosphingosine is N-hexanoyl-phytosphingosine.

3. The cosmetic formulation according to claim 1, wherein no further ceramides besides said N-acyl-phytosphingosine are present in said cosmetic formulation.

4. The cosmetic formulation according to claim 1, wherein said N-acyl-phytosphingosine is present in said cosmetic formulation in a concentration of 0.02% by mass to 2% by mass, based on the total cosmetic formulation.

5. The cosmetic formulation according to claim 1, wherein said 1,2-pentanediol is present in said cosmetic formulation in a concentration of 0.2% by mass to 15% by mass, based on the total cosmetic formulation.

6. The cosmetic formulation according to claim 1, wherein the ratio of N-acyl-phytosphingosine to 1,2-pentanediol is in the range from 1:100 to 1:1.

7. The cosmetic formulation according to claim 1, further comprising at least one C8/C10-glycerol partial ester.

8. A method of treating skin of a mammal, said method comprising:
applying a cosmetic formulation comprising an N-acyl-phytosphingosine, and 1,2-pentanediol to said skin.

9. (canceled)

10. (canceled)

11. The method according to claim 8, wherein said skin is present on a face of said mammal.

12. The method according to claim 8, wherein said skin is present on a breast of said mammal.

13. A method of reducing slackening of skin of a mammal, said method comprising:
applying a cosmetic formulation comprising an N-acyl-phytosphingosine to said skin.

14. The method according to claim 13, wherein said N-acyl-phytosphingosine is N-hexanoyl-phytosphingosine.

15. The method according to claim 14, wherein said N-hexanoyl-phytosphingosine is a sole ceramide present in said cosmetic formulation.

16. The method according to claim 13, wherein said skin is present on a face of said mammal.

17. The method according to claim 13, wherein said skin is present on a breast of said mammal.

18. A method of enlarging a breast of mammal, said method comprising:
applying a cosmetic formulation comprising N-hexanoyl-phytosphingosine to said breast.
